(19) United States
(12) Patent Application Publication
(10) Pub. No.: US 2023/0068453 A1
(43) Pub. Date: Mar. 2, 2023

(54) METHODS AND SYSTEMS FOR DETERMINING AND DISPLAYING DYNAMIC PATIENT READMISSION RISK AND INTERVENTION RECOMMENDATION

(71) Applicant: KONINKLIJKE PHILIPS N.V., EINDHOVEN (NL)

(72) Inventors: Gregory Boverman, Cambridge, MA (US); Eran Simhon, Cambridge, MA (US); David Paul Noren, Cambridge, MA (US); Lasith Adhikari, Cambridge, MA (US)

(21) Appl. No.: 17/884,698

(22) Filed: Aug. 10, 2022

Related U.S. Application Data
(60) Provisional application No. 63/236,725, filed on Aug. 25, 2021.

(51) Int. Cl. G16H 20/00; G16H 50/30; G16H 40/20

(52) U.S. Cl. CPC G16H 20/00; G16H 50/30; G16H 40/20

(57) ABSTRACT

A method for generating and presenting a patient readmission risk using a readmission risk analysis system, comprising: (i) receiving information about the patient comprising a plurality of readmission prediction features; (ii) extracting the plurality of readmission prediction features; (iii) generating an initial readmission risk for the patient for each of a first plurality of different future time periods; (iv) updating the plurality of readmission prediction features with one or more new readmission prediction features; (v) generating, by the trained readmission risk model using the one or more new readmission prediction features, an updated readmission risk; (vi) generating an intervention recommendation based on either the initial readmission risk or on the updated readmission risk for one or more of the plurality of different future time periods; and (vii) displaying a generated readmission risk and/or generated intervention recommendation.

### FIELD OF THE DISCLOSURE

The present disclosure is directed generally to methods and systems for predicting patient readmission risk and intervention using a readmission risk analysis system.

### BACKGROUND

Patient readmission occurs when a patient is readmitted to a hospital or other care facility within a certain period of time (e.g., days or weeks) after having been discharged, usually for treatment of the same or related condition. Readmission rates can be especially high for certain conditions. However, readmission can be unfavorable for several reasons. For example, high readmission rates may reflect upon the quality of the care provided by the hospital or care facility during the previous admission. Additionally, readmission can have significant financial consequences for the care facility. Indeed, readmission is becoming an increasingly important problem in the United States, due in large part to financial incentives and/or penalties from payers discouraging preventable readmissions.

Although high readmission rates can be unfavorable, it is often difficult to identify patients subject to readmission, and/or to identify the in-facility treatment or post-discharge care that best prevents readmission. It is therefore useful to predict readmission probability for a given patient, and to identify interventions both within and outside the hospital that could potentially reduce a patient's risk or need for readmission.

There has recently been interest in the development of machine learning algorithms for predicting hospital readmissions, particular as the Center for Medicare and Medicaid Services has instituted penalties for hospital readmissions within thirty days that are deemed preventable. While various models have shown some utility in reducing readmissions, they all suffer from significant limitations. For example, these methods have little or no use for physiological data acquired during a patient's hospital stay. Further, the methods do not utilize multiple future time periods when analyzing or predicting readmission risk, as existing readmission-prediction algorithms typically provide a risk assessment for a fixed time period, typically thirty days. Additionally, the methods fail to provide specific intervention recommendations.

### SUMMARY OF THE DISCLOSURE

Accordingly, there is a continued need for methods and systems that predict patient readmission risk for multiple future time periods and provide specific intervention recommendations. Various embodiments and implementations herein are directed to a method and system configured to generate and present a patient readmission probability or risk using a readmission risk analysis system. The system receives information about the patient, comprising a plurality of readmission prediction features. The system then extracts the plurality of readmission prediction features from the received information, and generates an initial readmission risk for the patient for each of a first plurality of different future time periods using a trained readmission risk model. The system can update the readmission prediction features with one or more new readmission prediction features received about the patient. Using these new readmission prediction features, the trained readmission risk model can generate an updated readmission risk for the patient for one or more of a second plurality of different future time periods. The trained readmission risk model generates an intervention recommendation based on either the initial readmission risk or on the updated readmission risk for one or more of the plurality of different future time periods, and provides the generated intervention recommendation via a user interface of the readmission risk analysis system.

Generally, in one aspect, a method for generating and presenting a patient readmission risk using a readmission risk analysis system is provided. The method includes: (i) receiving, at the readmission risk analysis system, information about the patient, wherein the information comprises a plurality of readmission prediction features; (ii) extracting, by a processor of the readmission risk analysis system, the plurality of readmission prediction features from the received information; (iii) generating, by a trained readmission risk model using the extracted plurality of readmission prediction features, an initial readmission risk for the patient for each of a first plurality of different future time periods; (iv) updating the plurality of readmission prediction features with one or more new readmission prediction features received about the patient; (v) generating, by the trained readmission risk model using the one or more new readmission prediction features, an updated readmission risk for the patient for one or more of a second plurality of different future time periods, where the different future time periods of the second plurality of different future time periods are the same or different from the different future time periods of the first plurality of different future time periods; (vi) generating, by the trained readmission risk model, an intervention recommendation based on either the initial readmission risk or on the updated readmission risk for one or more of the plurality of different future time periods; and (vii) displaying, via a user interface of the readmission risk analysis system, one or more of: the generated initial readmission risk for the patient for one or more of the first plurality of different future time periods; the generated updated readmission risk for the patient for one or more of the second plurality of different future time periods; and the generated intervention recommendation.

According to an embodiment, the method further includes the step of implementing the provided intervention recommendation.

According to an embodiment, the method further includes the step of training the readmission risk model of the readmission risk analysis system using historical patient data.

According to an embodiment, the patient's readmission risk is updated continually in real-time.

According to an embodiment, the one or more new readmission prediction features received about the patient are received after the patient is discharged.

According to an embodiment, the one or more new readmission prediction features received about the patient are received from a patient home monitoring device.

According to an embodiment, at least some of the information about the patient comprising the plurality of readmission prediction features is received from an electronic medical records database or system.

According to an embodiment, the method further includes the step of alerting a user if the generated updated readmission risk for the patient exceeds a predetermined threshold.

According to another aspect, a system for generating and presenting a patient readmission risk is provided. The system includes: a trained readmission risk model configured to generate a readmission risk from a plurality of extracted readmission prediction features about a patient; a processor configured to: (i) receive information about the patient, wherein the information comprises a plurality of readmission prediction features; (ii) extract the plurality of readmission prediction features from the received information; (iii) generate, using the trained readmission risk model, and the extracted plurality of readmission prediction features, an initial readmission risk for the patient for each of a first plurality of different future time periods; (iv) update the plurality of readmission prediction features with one or more new readmission prediction features received about the patient; (v) generate, using the trained readmission risk model and the one or more new readmission prediction features, an updated readmission risk for the patient for one or more of a second plurality of different future time periods, where the different future time periods of the second plurality of different future time periods are the same or different from the different future time periods of the first plurality of different future time periods; and (vi) generate, using the trained readmission risk model, an intervention recommendation based on either the initial readmission risk or on the updated readmission risk for one or more of the plurality of different future time periods; and a user interface configured to present to a user one or more of: (i) the generated initial readmission risk for the patient for one or more of the first plurality of different future time periods; (ii) the generated updated readmission risk for the patient for one or more of the second plurality of different future time periods; and (iii) the generated intervention recommendation.

According to an embodiment, the processor is further configured to generate an alert if the generated updated readmission risk for the patient exceeds a predetermined threshold.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for predicting patient readmission risk, in accordance with an embodiment.

FIG. 2 is a schematic representation of a readmission risk analysis system, in accordance with an embodiment.

FIG. 3 is a flowchart of a method for training a patient readmission risk model, in accordance with an embodiment.

FIG. 4 is a flowchart of a method for providing a readmission risk alert, in accordance with an embodiment.

FIG. 5 is a flowchart of a method for training a patient readmission risk model, in accordance with an embodiment.

FIG. 6 is a schematic representation of a readmission risk analysis system, in accordance with an embodiment.

### DETAILED DESCRIPTION OF EMBODIMENTS

The present disclosure describes various embodiments of a system and method configured to generate and present a patient's readmission risk. More generally, Applicant has recognized and appreciated that it would be beneficial to provide a method and system to prevent patient readmission. Accordingly, a trained readmission risk analysis system quantifies and communicates patient readmission risk to a clinician along with one or more intervention recommendations, which facilitates clinician decision-making for in-facility and/or post-discharge care for the patient. The trained readmission risk analysis system receives information about a patient, the information including a plurality of readmission prediction features. These features are extracted from the received information and the system then generates an initial readmission risk for the patient for each of a first plurality of different future time periods using a trained readmission risk model. The system can update the readmission prediction features with one or more new readmission prediction features received about the patient. Using these new readmission prediction features, the trained readmission risk model can generate an updated readmission risk for the patient for one or more of a second plurality of different future time periods. The trained readmission risk model generates an intervention recommendation based on either the initial readmission risk or on the updated readmission risk for one or more of the plurality of different future time periods, and provides the generated intervention recommendation via a user interface of the readmission risk analysis system. The system can also display one or more of the initial readmission risk and the updated readmission risk for the first or second plurality of different future time periods.

According to an embodiment, the systems and methods described or otherwise envisioned herein can, in some non-limiting embodiments, be implemented as an element for a commercial product for patient analysis or monitoring, such as the Philips® Patient Flow Capacity Suite (PFCS) (available from Koninklijke Philips NV, the Netherlands), or any suitable patient or care facility system.

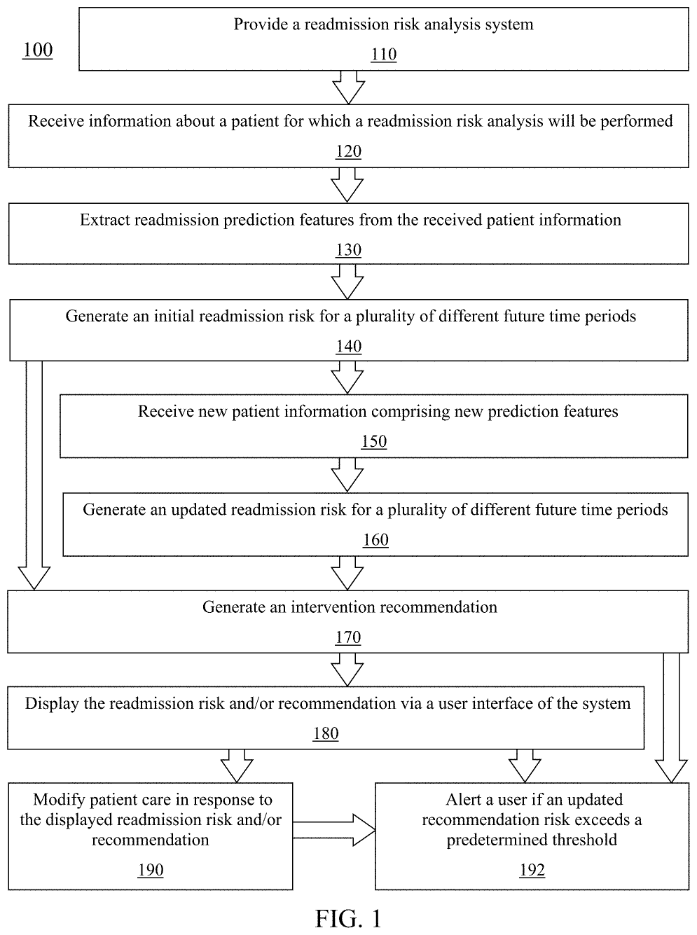

Referring to FIG. 1, in one embodiment is a flowchart of a method 100 for generating and communicating a patient readmission risk using a readmission risk analysis system. The methods described in connection with the figures are provided as examples only, and shall be understood not limit the scope of the disclosure. The readmission risk analysis system can be any of the systems described or otherwise envisioned herein. The readmission risk analysis system can be a single system or multiple different systems.

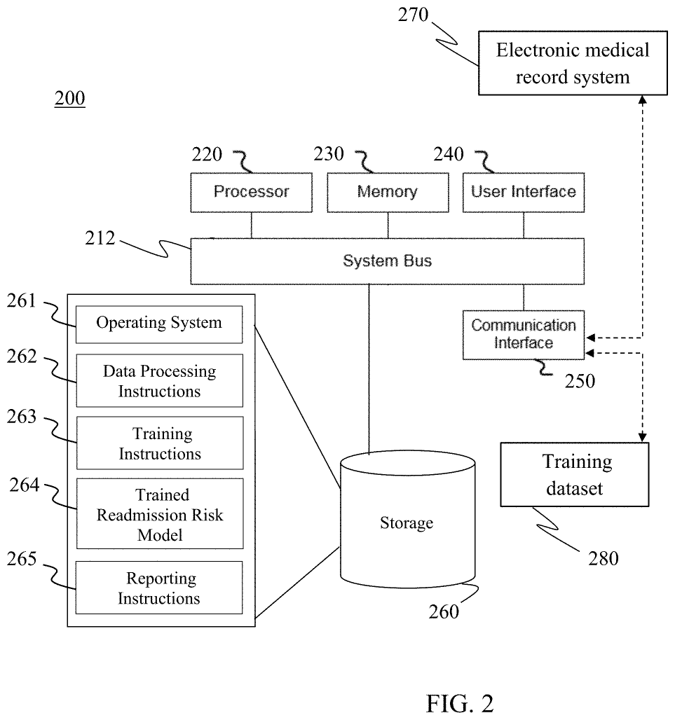

At step 110 of the method, a readmission risk analysis system is provided. Referring to an embodiment of a readmission risk analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, readmission risk analysis system 200 can be any of the systems described or otherwise envisioned herein. Other elements and components of readmission risk analysis system 200 are disclosed and/or envisioned elsewhere herein.

At step 120 of the method, the patient readmission risk analysis system receives information about a patient for which an analysis will be performed. According to an embodiment, the information comprises a plurality of features about the patient. Only some, or all, of the information about the patient and features may ultimately be utilized by the patient readmission risk analysis system. The plurality of features may comprise, for example, demographic and/or medical features about the patient. For example, demographic information or features may comprise age, gender, past healthcare facility visits or admissions, and other demographic information. Medical information or features may comprise vital sign information about the patient, including but not limited to physiologic vital signs such as heart rate, blood pressure, respiratory rate, apnea, SpO₂, invasive arterial pressure, noninvasive blood pressure, physiological measurements other than vital data such as physical observations, patient diagnosis or medication condition such as cardiac disease, psychological disorders, chronic obstructive pulmonary disease, and more, among many other types of medical information. Many other types of patient information are possible. Accordingly, the received information can be any information relevant to a patient readmission risk analysis.

The patient readmission risk analysis system can receive patient information from a variety of different sources, including any source that comprises one or more patient features. According to an embodiment, the patient analysis readmission risk analysis system is in communication with an electronic medical records database from which the patient information and one or more of the plurality of features may be obtained or received. The electronic medical records database may be a local or remote database and is in communication with the patient readmission risk analysis system 200. According to an embodiment, the patient readmission risk analysis system comprises an electronic medical record database or system 270 which is optionally in direct and/or indirect communication with system 200. According to another embodiment, the patient readmission risk analysis system may obtain or receive the plurality of features from equipment or a healthcare professional obtaining that information directly from the patient. According to another embodiment, the patient readmission risk analysis system may obtain or receive the plurality of features from home monitoring equipment, such as a wearable device, CPAP machine, or any other device utilized by a patient outside of a hospital or other direct healthcare facility.

According to an embodiment, the patient information includes patient demographics, clinical and operational information from multiple healthcare information systems, including electronic medical records (EMR), radiology information systems, cardiology information systems, lab information systems, and other systems. From these sources, the following types of information that are useful for risk estimation can be linked and collected: (i) patient demographic information (for example age, gender, race, height, weight, etc.); (ii) previous hospital visit operational information (for example time of appointment and visit, reason for visit, healthcare providers, etc.) within a previous time period such as a year although many other time periods are possible; (ii) current hospital visits clinical information (for example diagnosis, medical imaging and report, treatment, medication, etc.); (iii) medical history and family history; (iv) other information from other sources (for example geographic-based census information, socioeconomic information, personal survey) could also be leveraged to for analysis; and many other types or sources of patient information. According to an embodiment, the patient readmission risk analysis system may query an electronic medical record database or system, comprising fast healthcare interoperability resources (FHIR) for example, to obtain the patient information.

According to an embodiment, the patient readmission risk analysis system may comprise a data-ingestion or data-engineering module that maps hospital data to standardized covariates used in the readmission risk predictor and intervention recommender of the patient readmission risk analysis system.

The patient information received by the patient readmission risk analysis system may be processed by the system according to methods for data handling and processing/preparation, including but not limited to the methods described or otherwise envisioned herein. The patient information received by the patient readmission risk analysis system may be utilized, before or after processing, immediately or may be stored in local or remote storage for use in further steps of the method.

At step 130 of the method, the patient readmission risk analysis system extracts demographic and/or medical features from the patient information received in step 120 of the method. The features can be any of the features described or otherwise envisioned herein. The system can be trained to identify and extract the patient features from the received patient information, using any of a wide variety of algorithms, methods, or systems for identifying and extracting patient data. The plurality of identified and extracted patient features may be utilized immediately or may be stored in local or remote storage for use in further steps of the method. According to an embodiment, the patient information and/or patient features may undergo data processing at any stage.

At step 140 of the method, a trained model of the patient readmission risk analysis system analyzes the extracted readmission prediction features to generate an initial readmission risk for the patient for each of a first plurality of different future time periods. The trained model may be any module, algorithm, or machine learning method for analyzing input features and predicting readmission risk for a patient. The output of the trained model can be a predicted readmission risk, and this output may be utilized immediately or may be stored in local or remote storage for use in further steps of the method. Methods for training the readmission risk model are described or otherwise envisioned herein.

According to an embodiment, the patient is grouped into one or more of several different readmission time periods or classes, or the risk of the patient's readmission during the one or more different readmission time periods or classes is provided. For example, the readmission risk model or classifier can be trained to group the patient into one or more of—and/or identify patient readmission risk for each of—the following time periods or classes: (i) no readmission risk; (ii) readmission risk in fewer than five days; (iii) readmission risk between five days and thirty days; (iv) readmission risk between thirty days and sixty days. These time periods are non-limiting examples, and any other time periods are possible.

According to an embodiment, the patient readmission risk analysis system can generate specific alerts based on the predicted time windows for readmission. For example, if a clinician is considering discharging a patient and the risk of immediate readmission (i.e., less than five (5) days) is predicted to be high, the clinician may wish to reconsider. In contrast, for readmissions likely to occur over a longer time-horizon such as greater than thirty days, other interventions may be appropriate.

At step 150 of the method, the patient readmission risk analysis system receives new information about the patient, and updates the plurality of readmission prediction features with one or more new readmission prediction features extracted from the new information. The readmission risk for the patient can be updated continually or periodically in real-time using patient information, such that the risk is updated when new patient information is received. According to an embodiment, the patient is monitored for readmission risk after being discharged from a care facility such as a hospital, emergency department or urgent care facility. Accordingly, the one or more new readmission prediction features received about the patient can be received after the patient is discharged, although according to another embodiment the new features and the updated readmission risk can be received and generated, respectively, while the patient is admitted to a care facility such as a hospital, emergency department or urgent care facility. According to one embodiment, the one or more new readmission prediction features received about the patient are received from a patient home monitoring device. For example, the new patient information can be obtained from a patient device such as a wearable device, a thermometer, a blood pressure monitor, or any of a wide variety of other devices. The home monitoring device can be in direct or indirect wired and/or wireless communication with the patient readmission risk analysis system in order to transmit or otherwise communicate patient information to the system.

At step 160 of the method, the trained readmission risk model analyzes the one or more new readmission prediction features to generate an updated readmission risk for the patient for one or more of a plurality of different future time periods. Notably, these different future time periods can be the same time periods that were utilized for the initial readmission risk. Thus, the time periods can be the same time periods (such as less than five days and so on), or the dates that are covered by the time periods can be the same dates (i.e., less than five days includes the same five days in August, etc.). Alternatively, these different future time periods can be different time periods than those that were utilized for the initial readmission risk. Thus, the time periods can be different time periods (such as less than ten days, 11 to 15 days, etc.), or the dates that are covered by the time periods can be different dates (i.e., less than five days covers a different five-day period in August, etc.). The readmission risk for the patient can be updated continually or periodically in real-time using patient information, such that the risk is updated when new patient information is received.

According to an embodiment, generating an initial readmission risk and/or generating an updated readmission risk for the patient comprises generating, calculating, estimating, or otherwise determining an effect on the generated readmission risk of one or more of the individual readmission prediction features. For example, the readmission risk analysis system may determine a SHAP (SHapley Additive exPlanation) value or LIME score for one or more of the individual readmission prediction features. The SHAP value or LIME score may be determined using any method for determining SHAP values or LIME scores, or otherwise generating, calculating, estimating, or otherwise determining the effect. The SHAP values or LIME scores may be utilized immediately or may be stored in local or remote storage for use in further steps of the method.

At step 170 of the method, the trained readmission risk model of the readmission risk analysis system generates an intervention recommendation based in part or in whole on the initial readmission risk and/or on the updated readmission risk for one or more of the plurality of different future time periods. The intervention recommendation can also be based in part or in whole on the readmission prediction features utilized to generate the readmission risks.

According to an embodiment, the trained readmission risk model of the readmission risk analysis system comprises an intervention recommender. The intervention recommender can be composed of two parts. The first part can be an intervention set identifier which uses the readmission risk prediction to determine a set of potentially effective interventions for the patient. This can be accomplished, for example, by adding interventions that have not yet been considered to the data entered into the risk predictor to determine if the risk decreases, among many other approaches. The second part of the intervention recommender can be an intervention effectiveness filter that refines a list of potential interventions using patient profiles calculated from propensity score evaluation of predetermined patient group and intervention sets. According to an embodiment, based on readmission model outputs, the system can determine specific interventions as described or otherwise envisioned herein.

At step 180 of the method, the readmission risk analysis system displays or otherwise provides the generated readmission risk and/or intervention recommendation to a clinician or other user via a user interface. The display may comprise information about the patient, the input features, the generated initial readmission risk for the patient for one or more of the first plurality of different future time periods, the generated updated readmission risk for the patient for one or more of the second plurality of different future time periods, and/or the generated intervention recommendation. Other information is possible. Alternatively, the information may be communicated by wired and/or wireless communication to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands.

According to an embodiment, the display further includes information about an effect of one or more of the individual readmission prediction features in the complete set of readmission prediction features on the generated initial or updated readmission risk. According to an embodiment, the display may comprise a list, such as a ranked list, of one or more individual readmission prediction features with a highest effect on a higher readmission risk for the patient. For example, the display may comprise the top three individual readmission prediction features with the highest impact on the increased probability of readmission. According to an embodiment, the display may comprise a list, such as a ranked list, of one or more individual readmission prediction features with a highest effect on a lowered readmission risk for the patient. For example, the display may comprise the top three individual readmission prediction features with the highest impact on the decreased probability of readmission. Although three individual readmission prediction features are described in these examples, the actual number can be any number from zero to one to multiple features. According to an embodiment, determination of which individual readmission prediction feature(s) to display is based on comparing the effect of the prediction feature on a predetermined threshold, and displaying those features that meet and/or exceed that threshold. For example, a clinician may want to be notified of any prediction features that surpass a certain threshold, and thus this may be a setting or other variable parameter of the system.

According to an embodiment, the system can provide or display multiple possible intervention recommendations. According to an embodiment, the display may comprise a list, such as a ranked list, of one or more intervention recommendations.

At optional step 190 of the method, a clinician or other decision maker utilizes the displayed generated initial and/or updated readmission risk analysis, and/or the generated intervention recommendation, for patient care decision-making. For example, if the patient comprises a high readmission risk for a specific future time period based on the displayed information, the clinician or other decision maker can consider, order, or otherwise implement an intervention for the patient to address the high readmission risk. For example, the decision maker can consider or implement decisions such as the patient discharge disposition, including whether the patient is discharged to home, or a care facility, or to another facility. The decision maker can consider or implement decisions such as follow-up frequency and/or type for the patient, home visits, social care visits, monitoring at home, and/or a lot of other decisions. These decisions, and their implementation, can be an attempt by the clinician to affect—and preferable to lower—the readmission risk of the patient. The possible or potential effect of the decision on the readmission risk of the patient may be seen immediately or some period of time after the decision and/or implementation of the decision.

At optional step 192 of the method, the readmission risk analysis system alerts a user if an updated readmission risk for the patient exceeds a predetermined threshold. According to an embodiment, data can be collected in real time before and/or after discharge through an EMR. Any update in EMR data (e.g., medication, lab test results, missing appointment, etc.) can trigger the readmission algorithm to recalculate the readmission risk of the patient. If the difference between the initial patient readmission risk and the updated patient readmission risk is higher than a pre-defined threshold, then the care provider can be notified and/or a recommendation for intervention can be suggested. As just one non-limiting example, an increase of 10% could trigger a phone call, text message, or other communication to the patient, while an increase of 20% could trigger a home visit for the patient, although other thresholds are possible. According to an embodiment, changes in readmission risk can be visualized together with a reason for the change. In this way, the user can easily identify trends and understand what drives them.

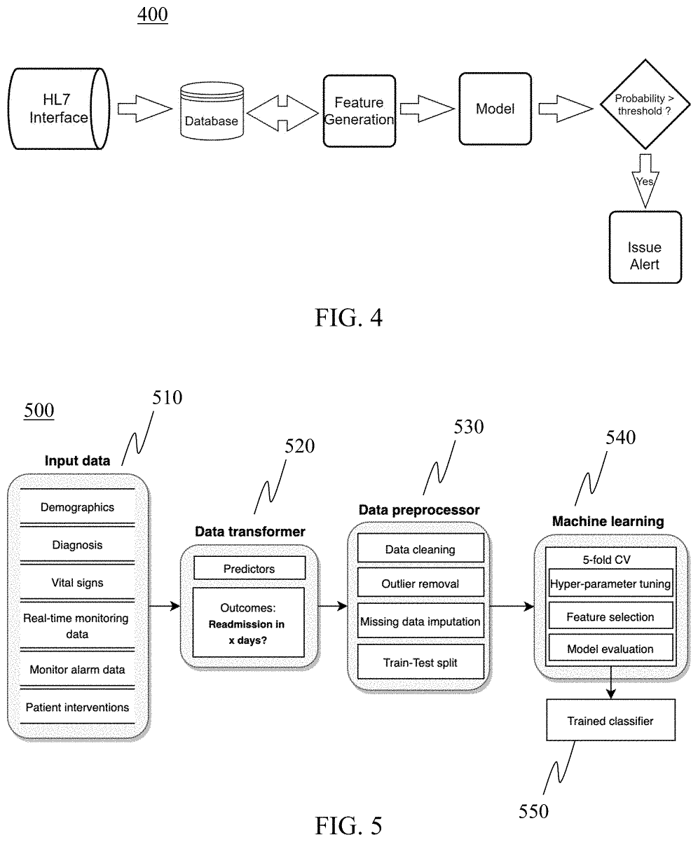

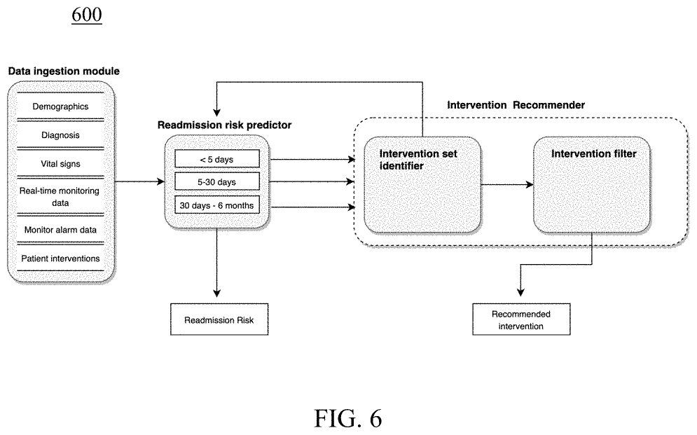

Referring to FIG. 4, in one embodiment, is a flowchart of a method 400 for alerting a system or user if an updated readmission risk for the patient exceeds a predetermined threshold. According to an embodiment, new patient information or data can be collected in real time before and/or after discharge through an EMR, such as through a medical data interchange format and standard like HL7 and FHIR. The received data may be utilized immediately or may be stored in a local or remote database for use in further steps of the method. The system can extract a plurality of readmission prediction features from the new patient information or data (“feature generation”) and the trained readmission risk model can generate an updated readmission risk for the patient. The system can then compare the updated readmission risk for the patient to the initial readmission risk for the patient and/or to a predetermined threshold, and can determine whether the updated readmission risk is higher than the predetermined threshold, and/or whether the change between the initial readmission risk and the updated readmission risk exceeds a predetermined change threshold. If the comparison reveals a change or risk above the predetermined threshold, the system can trigger an alert as described or otherwise envisioned herein. Accordingly, using real-time input from the electronic medical record (EMR), readmission outcomes are predicted using real-time medical data and social determinants of health (SDOH) data, issuing automated alerts when this readmission probability exceeds a certain threshold.

Referring to FIG. 2 is a schematic representation of a readmission risk analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, trained readmission risk model 264, and/or reporting instructions 265.

According to an embodiment, the electronic medical record system 270 is an electronic medical records database from which the information about the patient, including the plurality of readmission prediction features, may be obtained or received. The electronic medical records database may be a local or remote database and is in communication the patient readmission risk analysis system 200. According to an embodiment, the patient readmission risk analysis system comprises an electronic medical record database or system 270 which is optionally in direct and/or indirect communication with system 200.

According to an embodiment, the training data set 280 is a dataset that may be stored in a local or remote database and is in communication the patient readmission risk analysis system 200. According to an embodiment, the patient readmission risk analysis system comprises a training data set 280. The training data can comprise medical information about a patient, including but not limited to demographics, physiological measurements such as vital data, physical observations, and/or diagnosis, among many other types of medical information.

According to an embodiment, embodiment, data processing instructions 262 direct the system to retrieve and process input data which is used to train the readmission risk model 264. The data processing instructions 262 direct the system to, for example, receive or retrieve input data to be used by the system as needed, such as from electronic medical record system 270 among many other possible sources. As described above, the input data can comprise a wide variety of input types from a wide variety of sources, including home monitoring devices and other patient monitoring devices, among other devices and systems.

According to an embodiment, the data processing instructions 262 also direct the system to process the input data to generate a plurality of readmission prediction features related to medical information for a plurality of patients, which are used to train the classifier. This can be accomplished by a variety of embodiments for feature identification, extraction, and/or processing. The outcome of the feature processing is a set of readmission prediction features related to readmission risk analysis for a patient, which thus comprises a training data set that can be utilized to train the risk model 264.

According to an embodiment, training instructions 263 direct the system to utilize the processed data to train the readmission risk model 264. The readmission risk model can be any machine learning algorithm, classifier, or model sufficient to utilize the type of input data provided, and to generate a risk analysis. Thus, the system comprises a trained readmission risk model 264 configured to generate a readmission risk prediction for a patient, as described or otherwise envisioned herein. The trained readmission risk model 264 is also configured to generate one or more intervention recommendations as described or otherwise envisioned herein.

According to an embodiment reporting instructions 265 direct the readmission risk analysis system to generate and provide to a user via a user interface information comprising a generated initial and/or updated readmission risk, and/or the generated intervention recommendation. Alternatively, the information may be communicated by wired and/or wireless communication to another device. For example, the system may communicate the information to a mobile phone, computer, laptop, wearable device, and/or any other device configured to allow display and/or other communication of the information.

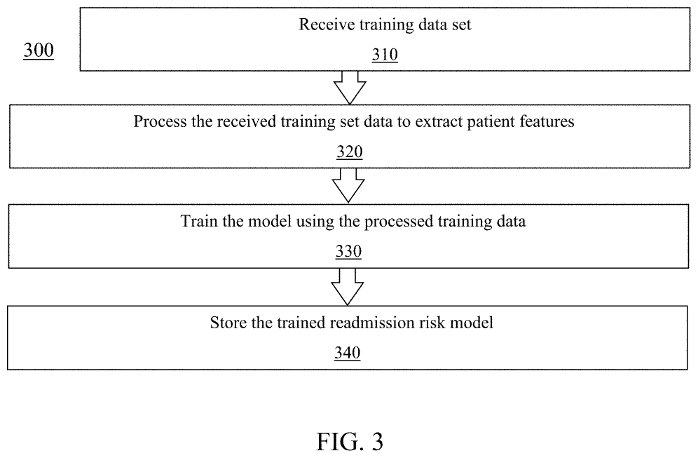

Referring to FIG. 3, in one embodiment, is a flowchart of a method 300 for training the readmission risk model of the patient readmission risk analysis system. At step 310 of the method, the system receives a training data set comprising training data about a plurality of patients. The training data can comprise medical information about each of the patients, including but not limited to demographics, physiological measurements such as vital data, physical observations, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible. According to an embodiment, the training data may also comprise an indication or information about one or more outcomes of each patient, including readmission information. According to an embodiment, the training data may comprise retrospective admission/discharge/transfer (ADT) data. The training data may be stored in and/or received from one or more databases. The database may be a local and/or remote database. For example, the patient readmission risk analysis system may comprise a database of training data.

According to an embodiment, the readmission risk analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract readmission prediction features about one or more of the plurality of patients. The readmission prediction features may be any features which will be utilized to train the readmission risk model 264, such as any readmission prediction features that can or will be utilized by the trained algorithm for readmission risk analysis for a future patient. Feature extraction can be accomplished by a variety of embodiments for feature identification, extraction, and/or processing, including any method for extracting features from a dataset. The outcome of a feature processing step or module of the readmission risk analysis system is a set of readmission prediction features about a plurality of patients, which thus comprises a training data set that can be utilized to train the classifier.

At step 330 of the method, the system trains the machine learning algorithm, which will be the algorithm utilized in analyzing patient information as described or otherwise envisioned. The machine learning algorithm is trained using the extracted features according to known methods for training a machine learning algorithm. According to an embodiment, the algorithm is trained, using the processed training dataset, to generate a readmission risk for the patient for each of a plurality of different future time periods, such as (i) no readmission risk; (ii) readmission risk in fewer than five days; (iii) readmission risk between five days and thirty days; (iv) readmission risk between thirty days and sixty days. These time periods are non-limiting examples, and any other time periods are possible. According to an embodiment, the algorithm is also trained, using the processed training dataset, to generate one or more intervention recommendations.

At step 340 of the method, the trained patient readmission risk analysis model is stored for future use. According to an embodiment, the model may be stored in local or remote storage.

Referring to FIG. 4, in one embodiment, is a flowchart of a method 500 for training the readmission risk model of the patient readmission risk analysis system. According to an embodiment, the system comprises both a readmission prediction algorithm and an intervention effectiveness filter configured to refine a calculated intervention list. Therefore, according to an embodiment, the system trains a machine learning classifier retrospectively using patient data mapped to specific outcomes, specifically readmission and readmission time. In one embodiment, the system groups the patients into several classes such as: (i) no readmission risk; (ii) readmission risk in fewer than five days; (iii) readmission risk between five days and thirty days; (iv) readmission risk between thirty days and sixty days. These time periods are non-limiting examples, and any other time periods are possible.

According to an embodiment, at step 510 of the method the patient readmission risk analysis system receives or obtains input data. The input data can comprise, among other data, detailed information about patient demographics (such as age, gender, etc.), diagnosis (such as comorbidities like cardiac disease, psychological disorders, chronic obstructive pulmonary disease, etc.), physiologic vital signs (such as heart rate, blood pressure, respiratory rate, oxygen saturation, etc.), real-time monitoring data, and monitor alarm data. The system can also collect data related to patient interventions (such as home visits, additional therapy, etc.).

At step 520 of the method, a data transformer of the system reads raw input data and generates predictor and outcome variables. In particular, according to an embodiment, the outcome variable is readmission to the hospital within a number of specified time windows.

At step 530 of the method, a data pre-processor analyzes the data. Data preprocessing can be performed using, for example, a set of automated rules. Specifically, the system can clean clinical variables using expert opinion and medical knowledge, and extreme values in physiological variables can be removed. Outlier detection and removal can be done by replacing, for example, the top and bottom 1% of data using random uniform values generated from the neighboring data. After generating the final feature set based on the knowledge on this space, the system can perform missing data imputation where missing nominal variables can be replaced with distinct missing category and missing continuous variables will be replaced by median values. Notably, data imputation may depend on the classification model).

At step 540 of the method, a machine learning classifier (such as Random Forest, XGBoost, etc.) can be used to learn the patient clinical phenotypes and their readmission rates, while also accounting for social determinants of health. All hyper-parameters in the machine learning classifier can be tuned using grid search technique with five-fold cross validation on 70% training data. All models can be validated using the remaining 30% test data and the final model parameters can be selected based on performance matrices such as area under receiver operating characteristic curve (AUC) and area under the precision-recall curve (AUCPR). This method results in the generation of a trained classifier at 550 of the method.

According to an embodiment, the system can determine specific patient profiles that are most likely to benefit from time horizon specific interventions using propensity scoring system. For example, the system can map intervention sets to readmission risk time horizons (<5 days, 5-30 days, etc.): The classifier described above can be used to evaluate the impact of different interventions on the readmission risk. This is done by modifying changeable features (i.e., simulating interventions not yet applied) of the machine learning model and re-predicting the readmission risk. For example, assume there is a feature that indicates whether the patient is subscribed to home-visits program. If a patient is not subscribed, then the algorithm will re-predict readmission risk, but will change this feature to “Yes”. In this way, the impact of a home-visit program on readmission risk can evaluated for that patient. The algorithm can repeat this exercise for all possible interventions and can provide a list of recommended interventions with an estimation of how each one will impact the readmission risk.

According to an embodiment, the system can map intervention effectiveness to specific patient profiles. To select the most effective of these potential interventions, the readmission outcomes (time horizons) can be used to stratify the patients in the training data. Within each of the time horizons, similarity profiles can be developed across the features specified for the readmission risk input data. To do so, cohorts that match these profiles and were prescribed specified interventions can be compared with cohorts that did not receive the intervention. First, an initial propensity score can be computed to each cohort and each intervention. If the initial propensity score is too low or too high (meaning that the two cohorts differ by other factors beside the intervention) then the algorithm can remove patients from the intervention cohort until the propensity score satisfies a pre-defined threshold. This can be done by removing patients with the highest probability of belonging to each of the cohorts. Finally, the risk of readmission can be re-computed for each cohort. If the readmission score is less for the intervention cohort, aggregate feature values from that cohort can be used to define the patient profile. The weighting for the intervention effectiveness for that profile can be determined as a function of the reduction in readmission risk and the final propensity score.

Accordingly, the system can predict readmission over multiple time-horizons, using potentially different features for each window. Additionally, the system can compute a continuous readmission score that is updated in real-time by a hospital patient's electronic medical record data. For example, it is very likely that patient vital signs, laboratory values, and medications, will impact the risk of readmissions that occur within a short time following discharge, e.g. 1-5 days. In contrast, demographic and social factors are more likely to play a vital role in readmissions that occur after a longer time period, e.g., 20-30+ days.

Additionally, the system can use offline and retrospective reinforcement learning methods to suggest the most effective interventions to prevent readmission for a given patient. For example, the effectiveness of an intervention is not limited to the readmission rate alone, but can be defined across multiple dimensions. For instance, there is a balance between keeping patients in the hospital to prevent readmission and incurring excessive expense due to prolonged length-of-stay. An objective analysis can determine the strategy that best conserves cost while not sacrificing patient safety.

Referring to FIG. 6, in one embodiment, is a readmission risk analysis system 600 for generating and presenting a patient readmission risk and/or intervention recommendation. The system comprises a data-ingestion or data-engineering module that maps hospital data to standardized covariates used in the readmission risk predictor and intervention recommender of the patient readmission risk analysis system. The data-ingestion or data-engineering module receives a wide variety of different patient information. The patient information received by the patient readmission risk analysis system may be utilized, before or after processing, immediately or may be stored in local or remote storage for use in further steps of the method.

According to an embodiment, the readmission risk analysis system comprises a readmission risk predictor configured or trained to generate an initial and/or updated readmission risk for a plurality of different time periods.

According to an embodiment, the readmission risk analysis system comprises an intervention recommender that comprises an intervention set identifier which uses the readmission risk prediction to determine a set of potentially effective interventions for the patient, and an intervention effectiveness filter that refines a list of potential interventions using patient profiles calculated from propensity score evaluation of predetermined patient group and intervention sets. The intervention recommender can generate one or more intervention recommendations for one or more of the plurality of different time periods.

According to an embodiment, the patient readmission risk analysis system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the patient readmission risk analysis system. Thus, generating a functional and skilled trained classifier comprises a process with a volume of calculation and analysis that a human brain cannot accomplish in a lifetime, or multiple lifetimes.

In addition, the patient readmission risk analysis system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime.

By providing an improved patient readmission risk analysis, this novel patient readmission risk analysis system has an enormous positive effect on patient readmission risk analysis compared to prior art systems. As just one example in a clinical setting, by providing a system that can improve patient readmission risk analysis with confidence intervals, the system can facilitate treatment decisions and improve survival outcomes, thereby leading to saved lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating and presenting a patient readmission risk using a readmission risk analysis system, comprising:
receiving, at the readmission risk analysis system, information about the patient, wherein the information comprises a plurality of readmission prediction features;
extracting, by a processor of the readmission risk analysis system, the plurality of readmission prediction features from the received information;
generating, by a trained readmission risk model using the extracted plurality of readmission prediction features, an initial readmission risk for the patient for each of a first plurality of different future time periods;
updating the plurality of readmission prediction features with one or more new readmission prediction features received about the patient;
generating, by the trained readmission risk model using the one or more new readmission prediction features, an updated readmission risk for the patient for one or more of a second plurality of different future time periods, where the different future time periods of the second plurality of different future time periods are the same or different from the different future time periods of the first plurality of different future time periods;
generating, by the trained readmission risk model, an intervention recommendation based on either the initial readmission risk or on the updated readmission risk for one or more of the plurality of different future time periods; and
displaying, via a user interface of the readmission risk analysis system, one or more of: (i) the generated initial readmission risk for the patient for one or more of the first plurality of different future time periods; (ii) the generated updated readmission risk for the patient for one or more of the second plurality of different future time periods; and (iii) the generated intervention recommendation.

2. The method of claim 1, further comprising the step of implementing the provided intervention.

3. The method of claim 1, further comprising the step of training the readmission risk model of the readmission risk analysis system using historical patient data.

4. The method of claim 1, wherein the patient's readmission risk is updated continually in real-time.

5. The method of claim 1, wherein the one or more new readmission prediction features received about the patient are received after the patient is discharged.

6. The method of claim 1, wherein the one or more new readmission prediction features received about the patient are received from a patient home monitoring device.

7. The method of claim 1, wherein at least some of the information about the patient comprising the plurality of readmission prediction features is received from an electronic medical records database or system.

8. The method of claim 1, further comprising the step of alerting a user if the generated updated readmission risk for the patient exceeds a predetermined threshold.

9. A system for generating and presenting a patient readmission risk, comprising:
a trained readmission risk model configured to generate a readmission risk from a plurality of extracted readmission prediction features about a patient;
a processor configured to: (i) receive information about the patient, wherein the information comprises a plurality of readmission prediction features; (ii) extract the plurality of readmission prediction features from the received information; (iii) generate, using the trained readmission risk model, and the extracted plurality of readmission prediction features, an initial readmission risk for the patient for each of a first plurality of different future time periods; (iv) update the plurality of readmission prediction features with one or more new readmission prediction features received about the patient; (v) generate, using the trained readmission risk model and the one or more new readmission prediction features, an updated readmission risk for the patient for one or more of a second plurality of different future time periods, where the different future time periods of the second plurality of different future time periods are the same or different from the different future time periods of the first plurality of different future time periods; and (vi) generate, using the trained readmission risk model, an intervention recommendation based on either the initial readmission risk or on the updated readmission risk for one or more of the plurality of different future time periods; and
a user interface configured to present to a user one or more of: (i) the generated initial readmission risk for the patient for one or more of the first plurality of different future time periods; (ii) the generated updated readmission risk for the patient for one or more of the second plurality of different future time periods; and (iii) the generated intervention recommendation.

10. The system of claim 9, wherein the processor is further configured to receive input via the user interface comprising an implementation of the provided intervention recommendation.

11. The system of claim 9, wherein the patient's readmission risk is updated continually in real-time.

12. The system of claim 9, wherein the one or more new readmission prediction features received about the patient are received after the patient is discharged.

13. The system of claim 9, wherein the one or more new readmission prediction features received about the patient are received from a patient home monitoring device.

14. The system of claim 9, wherein at least some of the information about the patient comprising the plurality of readmission prediction features is received from an electronic medical records database or system.

15. The system of claim 9, wherein the processor is further configured to generate an alert if the generated updated readmission risk for the patient exceeds a predetermined threshold.
